(19) United States
(12) Patent Application Publication
(10) Pub. No.: US 2019/0060538 A1
(43) Pub. Date: Feb. 28, 2019

(54) METHODS AND DEVICES FOR ACCESS TO HYPER-INFLATED LUNG

(71) Applicant: SOFFIO MEDICAL INC., Boston, MA (US)

(72) Inventors: Mark GELFAND, New York, NY (US); Jianmin LI, Lexington, MA (US); George BOURNE, Boston, MA (US); Howard LEVIN, Teaneck, NJ (US); Benjamin David BELL, Shrewsbury, MA (US); Gerhard A. FOELSCHE, Rehoboth, MA (US); Aaron SANDOSKI, Boston, MA (US)

(21) Appl. No.: 15/772,516

(22) PCT Filed: Oct. 31, 2016
(86) PCT No.: PCT/US2016/059735

§ 371 Date: Apr. 30, 2018

Related U.S. Application Data
(60) Provisional application No. 62/249,008, filed on Oct. 30, 2015.

(51) Int. Cl. A61M 1/04; A61M 25/00; A61M 39/02; A61M 39/20

(52) U.S. Cl. CPC A61M 1/04; A61M 25/0074; A61M 39/0247; A61M 2039/0276; A61M 2205/04; A61M 2039/0252; A61M 39/20

(57) ABSTRACT

Devices to improve lung function in a patient having restricted ventilation. The device may include an entry or access port for an implantable airway bypass device that relieves trapped air. The device of the present invention comprises an expandable structure (164) to be implanted in lung tissue and defining an air capture chamber, a first conduit (174) having a first end from which extends the expandable structure, and a second end to be coupled to a first end of a second conduit (161), the second end of the second conduit being joined to an external anchor (175) to rest against an outer skin surface of the chest of the patient.

### RELATED APPLICATION

Noon This application claims priority to U.S. Provisional Patent Application 62/249,008 filed Oct. 30, 2015, the entirety of which is incorporated by reference.

### BACKGROUND

The present disclosure is directed generally to implantable medical devices to improve chest mechanics in diseased patients by forming and partially bypassing airways of the lung. The methods and devices disclosed herein may be configured to create alternative expiratory passages for air trapped in the emphysematous lung by establishing communication between the alveoli and/or other spaces with trapped air and the external environment thereby draining and reducing the hyperinflation of the lung parenchyma. Improvements over previous devices may include less invasive treatment options, avoidance of surgery and large area pleurodesis, minimization of disturbances and irritation of lung tissue to minimize inflammation or damage to untargeted areas of the lung and chest, better control of healing processes, and establishing long-term patency of artificial air passages.

Diseases of the lung such as Chronic Obstructive Pulmonary Disorder (COPD), emphysema, chronic bronchitis, and asthma may manifest with abnormally high resistance to airflow in an air pathway of the respiratory system. Homogeneous obstructive lung disease, also known as diffuse lung emphysema, is particularly difficult to treat and currently has few treatment options. Patients with pulmonary emphysema are unable to exhale appropriately, which leads to lung hyperinflation, which involves air trapping or excessive residual volume of air trapped in at least a portion of the lungs. The debilitating effects of the hyperinflation are extreme respiratory effort, the inability to conduct gas exchanges in satisfactory proportions, severe limitations of exercise ability, and a sensation of dyspnea and associated anxiety. Although optimal pharmacological and/or other medical therapies work well in the earlier stages of the disease, as it progresses, these therapies become increasingly less effective. For these patients, the standard of care is surgical treatment involving lung volume reduction surgery, lung transplantation or both.

It has been observed in prior art and is generally accepted by clinicians that respiratory impairment in emphysema has an important ‘mechanical’ component. Destruction of pulmonary parenchyma causes compounding disadvantages of a decreased mass of functional lung tissue decreasing the amount of gas exchange, and a loss in elastic recoil and hence the inability to equally or substantially completely exhale the same amount of air that was inhaled on the previous breath. This leads to the typical hyper-expansion of the chest with a flattened diaphragm, widened intercostal spaces, and horizontal ribs, resulting in increased effort to breath and dyspnea. When the destruction and hyper-expansion occur in a non-uniform manner, the most diseased lung tissue can expand to crowd the relatively less diseased or even normal lung tissue further reducing lung function by preventing optimal ventilation of the less diseased or normal lung. Lung volume reduction surgery (LVRS) and the surgical removal of the most affected lung regions conceptually would allow the relatively spared part of the remaining lung to function in mechanically improved conditions.

Some methods and devices propose making use of the non-uniform parenchymal destruction and the related lung mechanics found in emphysema. An opportunity may be presented in which the potential removal of the parts of the lung most effected by the disease allows the remaining lung to function normally. (e.g., expand in a satisfactory manner, and improve the overall elastic recoil of the chest cavity.) Unfortunately, these approaches have met with difficulties in long term device performance and viability.

In addition to internal complications (e.g. unintended tissue ingrowth, occlusion by naturally occurring secretions, effects resulting from heightened pro-inflammatory state, excessive bleeding, complications from device delivery, or eventual device rejection by the body) challenges also exist when delivering, deploying, sealing and securing an implantable airway bypass device. There is, therefore, a need for a medical device for novel methods and devices for facilitating the delivery and use of an airway bypass device.

### SUMMARY

Systems, methods and devices have been conceived and are disclosed herein for improving the mechanics of a diseased lung of a patient by implanting one or more airway bypass ventilation devices in a lung. For example, the patient may suffer from COPD, emphysema, chronic bronchitis, or asthma. An airway bypass device may create a connection between the lung parenchyma affected by abnormally high resistance airways to the atmosphere.

A device to allow the bypass of the airways of a diseased lung through minimally invasive implantation has been conceived and is disclosed herein. The device comprises an air intake component, and a relatively large surrounding expandable structure configured to hold the air intake component within a space in lung tissue created and occupied by the expandable structure. The device further comprises a conduit that approximately spans the distance of the chest wall. The conduit may comprise at least one hollow lumen fluidly connecting the intake component and space occupied by the expandable structure, allowing fluid to escape to an area of lower pressure.

The devices and methods may be configured to delivery and deploy the airway bypass device, while mitigating the risks from associated complications and minimizing tissue damage and inflammation. Once the device is delivered and deployed, it may create a space within the lung, connecting that space to a larger volume of the lung through mechanisms including collateral ventilation, and providing an airway bypass pathway from the space to a lower-pressure space (e.g. atmosphere). Air flow may naturally occur if flow resistance of the airway bypass pathway is sufficiently low.

One of several challenges facing the use of the device is the delivery and deployment of the relatively large structure using a minimally invasive approach. Methods and devices for creating an incision and delivery location may allow for the negotiation of multiple layers of lung tissue and pleurae, while minimizing risks (e.g. pneumothorax, localized trauma, implant rejection, device failure, etc.). The installation of an external anchor and the use of this aperture as an external access port may also be advantageous. Finally, the additional advantages of securing the device, formation and maintenance of a hermetic seal and prevention of unauthorized device access may also be described herein. These and other features and advantages of the invention will be apparent to those skilled in the art from the following detailed description, taken together with the accompanying drawings.

### BRIEF DESCRIPTION OF THE DRAWINGS

FIG. 1 is a combined illustration of chest anatomy showing the basic schematics of an airway bypass device implanted in a lung.

FIG. 2 shows the portion of an implant airway bypass device that spans the chest wall, forming a hermetic seal over multiple layers of tissue or pleurae.

FIG. 3 is an exploded view of the airway bypass device, showing the external components interfacing with the conduit and associated structures of the device.

FIG. 4A show the front view of a suture-mounted anchor on the exterior or skin of a patient.

FIG. 4B shows the cross sectional view a suture-mounted anchor on the exterior or skin of a patient.

FIG. 5A show the front view of a skin-mounted anchor on the exterior of a patient.

FIG. 5B shows the cross sectional view a skin-mounted anchor on the exterior of a patient.

FIG. 6 shows a patient-mounted airway anchor and cap, forming the external components of the device.

FIGS. 7A-D show multiple tamper-safe locking mechanisms or caps that may help to prevent unauthorized device access.

FIG. 8 shows a tamper-resistant locking mechanisms or caps formed of a mesh material.

FIG. 9A is a schematic showing additional external features.

FIG. 9b is a schematic showing a subcutaneous fluid reservoir, septum and access.

FIGS. 10-16 shows at least one incision and suturing technique that may be used to minimize the risk of pneumothorax during device delivery (i.e. positioning of the airway bypass device within the lung).

FIGS. 17-26 show a sequence of frames illustrating at least one technique for device delivery of a lung airway bypass device.

FIGS. 27-28 continue the sequence of frames illustrating the delivery technique and show device deployment following delivery.

FIGS. 29-34 continue the sequence of frames illustrating the delivery technique and show the delivery and installation an optional external anchor.

### DETAILED DESCRIPTION

Complexities may arise in the use of an airway bypass device resulting from the limitations of minimally invasive techniques combined with the dimensional requirements needed to create and maintain fluid communication between the internal lungs and the atmosphere. As such, novel systems, methods and devices for delivery and deployment are described herein. These systems, methods and devices may dramatically improve the mechanics of a diseased lung of a patient.

The reduction of lung hyper inflation may prove critical to relieving the symptoms of certain serious lung diseases (including COPD, emphysema, etc.) that may be characterized by slow or inefficient flow of gas into and out of the lung. Trapped air may increase with each new breaths before the exhalation of substantially all of the air inhaled on the previous breath. An abnormally high amount of air is withheld in the lung, for example in the alveoli and alveoli ducts and bronchioles. These small air filled cavities are within the smallest divisions of the lung and form areas of increased resistance to airflow that result in reduced lung expiration. Creation and maintenance of fluid communication from the internal lung to the atmosphere, facilitated by an airway bypass device, enables the improvement of the mechanics of a diseased lung. This ventilation allowing entire lobes or entire lung to empty trapped air through one or more artificial channels.

In many aspects, the minimally invasive implantation of an airway bypass device may be preferred to shorten recovery times, reduce trauma and lessen discomfort, as compared to conventional surgery. However, the dimensional limitation of minimally invasive approaches runs contrary to the use of certain advantageous airway bypass devices with relatively large internal volumes that act to reduce airway occlusion and dramatically improve device patency over the lifetime of the implant. Additionally, all surgical approaches that perforate the pleurae of the lung place a patient at greater risk of complications from the formation of a pneumothorax.

To allow device delivery and deployment within these dimensional limitations, while also minimizing the risk of pneumothorax, it is envisioned that the systems, methods and devices may employ various techniques for holding the lung in place over the course of delivery. The device may then be partially delivered and deployed with an external anchor subsequently attached and mounted on the surface of delivery location to secure the device and reforming the natural hermetic seal that naturally separates the lungs from the external atmosphere.

To deliver and secure the envisioned airway bypass device in an effective anatomical position. (e.g. specific portion of a patient's lung, upper lobe of a lung, other lobes of the lung), the location may be chosen for placement of the device (including the external portions of the device) based on factors such as low tissue density, low blood flow, trapped air, presence of a bulla, or depth. Based on device location, the fluid connection between the external atmosphere 50 and the internal lung 100 may also be used to deliver drugs, such as bronchodilators, to the distal-most areas of the lung considered the most inaccessible, but where such drugs would be most effective.

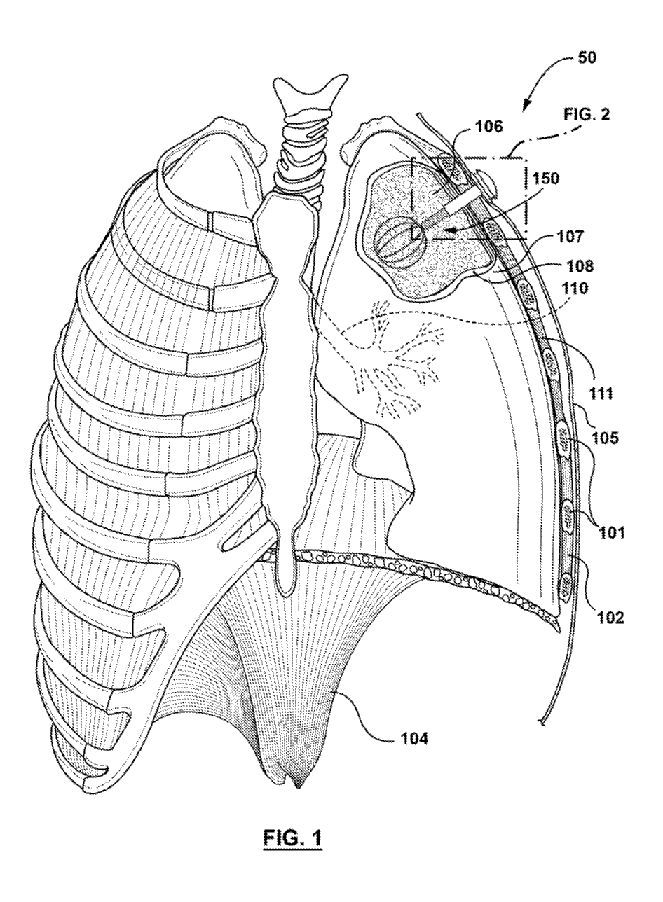

FIG. 1 represents a schematic illustration of various layers of a patient's rib cage and thoracic cavity. Beneath the skin 105 is a rib cage formed by a vertebral column, ribs 101, and sternum 103. The rib cage surrounds a thoracic cavity, which contains structures of the respiratory system including a diaphragm 104, trachea 109, bronchi 110 and lungs 100. An inhalation is typically accomplished when the muscular diaphragm 104, at the floor of the thoracic cavity, contracts and flattens, while contraction of intercostal muscles 102 lift the rib cage up and out. These actions produce an increase in volume, and a resulting partial vacuum, or negative pressure, in the thoracic cavity, resulting in atmospheric pressure pushing air into the lungs 100, inflating them. In a healthy person, an exhalation results when the diaphragm 104 and intercostal muscles 102 relax, and elastic recoil of the rib cage and lungs 100 expels the air.

In a patient with a disease such as COPD, emphysema, or chronic bronchitis a restriction in air pathways may cause resistance to air flow and impede the ability of air to be expelled, in at least a portion of the lungs 100, upon muscle relaxation and elastic recoil of the rib cage. The inability to expel air from the restricted portion of the lung may result in a need for increased physical exertion to expel the air, increased residual volume, barrel chest syndrome, or feelings of dyspnea and anxiety. Lung parenchyma 106 is the tissue of the lung 100 involved in gas transfer from air to blood and includes alveoli, alveolar ducts and respiratory bronchioles.

In human anatomy, the pleural cavity 134 is the potential space between the two pleurae 107, 108 of the lungs, namely the visceral 108 and parietal 107 pleurae. A pleura is a serous membrane which folds back onto itself to form a two-layered membrane structure. The area between the two pleural layers is known as the pleural cavity and normally contains a small amount of pleural fluid. The outer parietal pleura is attached to the chest wall. The inner visceral pleura covers the lungs and adjoining structures, via blood vessels, bronchi and nerves.

The pleural cavity, or pleural space, with its associated pleurae 107, 108, aids in the optimal functioning of the lungs during breathing. The pleural cavity 134 also contains pleural fluid, which allows the pleurae 107, 108 to slide effortlessly against each other. Surface tension of the pleural fluid also leads to close apposition of the lung surfaces with the chest wall. In addition, to function optimally, the pleural cavity 134 is maintained at a negative pressure. This combination of factors allows for inflation of the alveoli during breathing. The pleural cavity 134 transmits movements of the chest wall to the lungs, particularly during heavy breathing. This occurs because the closely apposed chest wall transmits pressures to the visceral pleural surface and hence to the lung.

Therapy would comprise the connection the lung parenchyma 106 to the atmosphere 50 by passing through both layers of pleura. As such, a conduit 161 that contains at least one hollow lumen 169 of an airway bypass device 150 is implanted into the lung parenchyma 106. The bypass device spans the chest wall and terminates at a position on the external surface of the patient's skin 105. The intake component is connected to the surface by a conduit 161 that passes out of the lung through a fused region (i.e. pleurodesis 112) between the visceral pleura 108 and parietal pleura 107, passes beneath the skin 105 and exits the skin.

Air or other fluids may pass through from the internal lung and exit the device externally. A flow of air may be created by a pressure differential between a higher-pressure region in the lung to a lower-pressure terminus of the device conduit 161, which may be atmosphere. Any pressure differential may be increased by further reducing pressure in the direction of fluid or air flow through the device, with the use of a number of external devices, for example with a pump. To simplify the connectivity of the device, for example to external medical devices, it is envisioned that the external terminuses or components the device may be connected to fittings including luer connectors.

The conduit 161 of the airway bypass device 150 connects to air intake component of the device. The conduit 161 may pass directly out of the chest wall, or the conduit 161 may pass beneath the skin a distance before being connected to the external atmosphere 50. Containing the device within the skin may help to reduce risk of infection in tissue around the device. Not illustrated, but also envisioned, is the termination of a conduit 161 in other expiratory areas of the body. In addition, it is envisioned that the conduit 161 may pass to a fluid reservoir that is held subcutenously under the skin, which would be accessible in certain embodiments of the device. The conduit 161 may be an elongate tube with at least one lumen 169 in communication with the air intake component 165 (e.g., via a lumen of a strain relief member) and the opposite terminus and may be made of a biocompatible flexible material such as silicon, Pebax or other polymer. The lumen 169 may be used for example for passage of air, fluid, catheters, replaceable sleeves, removable air intake catheters, or endoscopes. Multiple lumens may be present in the conduit 161—for example, a second lumen 169 may connect the device to the lymphatic system to drain collected fluid. In some instance, as needed, a replaceable inner sleeve may be inserted into the lumen 169 of the conduit 161 to clean the passageway, for example to remove biofilm that may form within the sleeve over time.

While fluid connection between the internal lung and the external atmosphere 50 is desired, it is important to also seal the pleural cavity 134 and space from communication with the atmosphere, as exposure could increase the risk of serious complications. Unfortunately, during device delivery, the creation of one or more incisions (at an delivery location), which exposes the pleural cavity 134 to the atmosphere 50 will unnaturally pressurize the pleural space surrounding the lungs, which may cause the intrapleural (i.e. pleural space) pressure to equal or exceed atmospheric pressure. This may result in the migration (i.e. collapse) of the lung, the first signs of a progressing pneumothorax or tension pneumothorax.

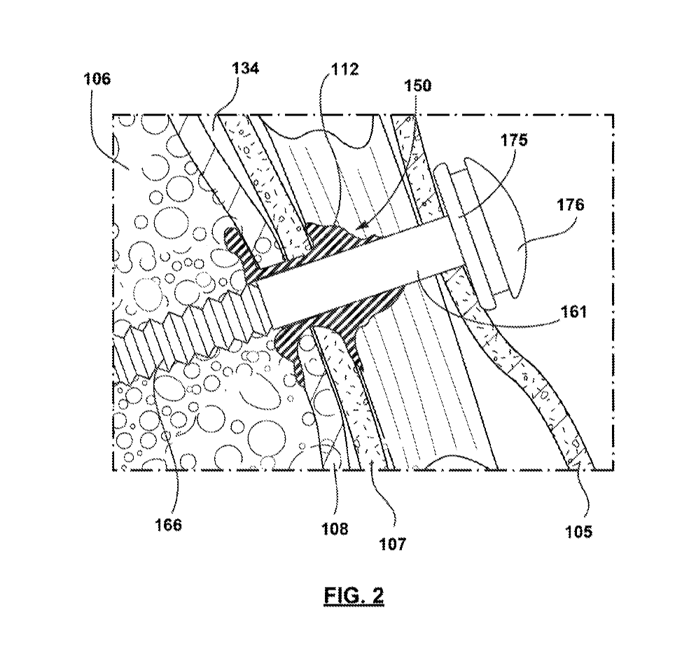

To reduce the occurrence of a pneumothorax, it may be desired that the parietal and visceral pleura be brought into contact, as illustrated in FIG. 2, to undergo localized pleurodesis 112. It is understood that the size and shape of this area may vary from patient to patient. The device conduit that connects the internal lung to the external atmosphere 50 may be surrounded by an access port that extends from the structure of the basket. This access port may be flexible and comprise a strain relief member, or may be rigid. In addition, to facilitate tissue ingrowth and accelerate the fusion of the pleura and formation of a local pleurodesis 112, it is envisioned that an ingrowth cuff may also surround the access port of the device.

While it is generally preferred that the formation of pleurodesis 112 avoids irreversibly fusing large areas of the pleura, the integrity of the seal between the atmosphere 50 and the pleural space is critical to the effectiveness of the device. In one or more of the embodiments described for forming local pleurodesis 112, it may be advantageous to use a tissue glue or dermal adhesive (e.g. lung sealant, a soft tissue glue) injected between the device, pleura or skin surface to enable adhesion at a relatively low contact pressure and to enable the rapid formation of an initial seal. Tissue glue or dermal adhesive 146 may also be used in combination with sutures 118 or other closure mechanisms to provide additional adherence and sealing to the access port.

In other embodiments, or in combination with pleurodesis 112 formation, it is envisioned that the migrating pleurae may be biased towards the chest wall to prevent the collapse of the lung. This biasing force may be achieved and maintained by way of sutures 118 (e.g. purse string or mattress sutures 118) that would seal the parietal and visceral pleura. Additionally, this sealed area may be connected at the delivery location 114 to an externally mounted access port. One or more suture pledgets 122 may be used to distribute the force of the sutures 118 across tissue and reduce the risk of perforation of the pleura. In addition to sutures 118, it is envisioned that alternate modes of artificial fixation may also be provided and includes dermal adhesives 146, hooks, staples, brackets or other features. As alternatives or in combination, the natural fixation of the lung though the formation of a local pleurodesis 112 is also envisioned.

In these embodiments, once sutures 118 have been thrown to prevent the lung pleurae 107, 108 from migrating away from a first incision on the chest tissue, a second incision can be made to perforate the pleurae, 107, 108 thereby creating a fluid connection and access to a delivery location 114. Once the first incision 116 has been made to create the delivery location 114, a wire 124, dilator 126, cannula, or dilator sheath 128 may be advanced through the chest wall and into the lung 100 (e.g., the wire, dilator, cannula, or dilator sheath may have increasing diameters of up to approximately 30 FR to pass through the intercostal space) to define and maintain a passage into the lung at the implant location. The device, in its undeployed configuration may then be delivered through the passage and into the lung. Exemplary methods of device delivery following the biasing of the lung to the chest wall are discussed further herein. To reduce the dimensions needed for delivery, it is envisioned in at least one embodiment that expandable structure 164 may be held in this undeployed configuration using a delivery sheath 120.

Once the internal components (e.g. the basket and access port) of the device have been positioned, the expandable structure may be deployed. It may be desirable that the expandable structure 164 be sufficiently compliant and flexible that it is able to freely or partially float and expand and contract along with movement by lung parenchyma (i.e. when breathing or coughing), thus minimizing friction, rubbing and potential tearing between lung parenchyma and the expanding structure, which may minimize or avoid irritation of the tissue, decreasing the risk of excessive and prolonged inflammation, scar formation or tissue regrowth. In addition, the expandable structure 164 would also be able to rise and fall with the natural respiratory motion of the lung. This additional flexure would be provided by the pliability of an optional strain relief member 166 in addition to the inherent pliability of the expandable structure 164.

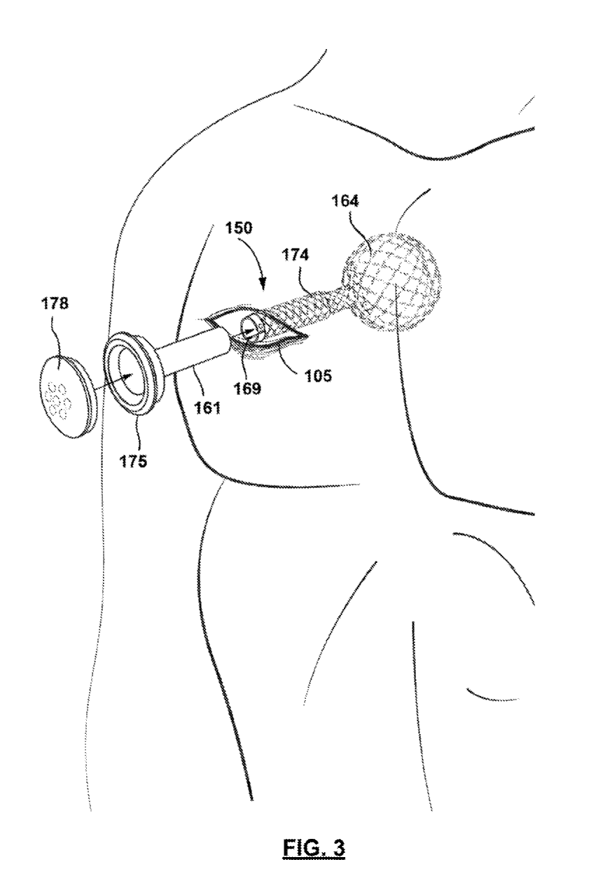

Subsequently, it is envisioned that one or more externally mounted features may be used to secure the device. As shown in FIG. 3, the access port may include a generally cylindrical conduit 174 extending from the expandable structure 164 through the pleura to be externally accessible. The port may be dimensioned to terminate just prior to the surface of the skin, may be flush to the surface or may extend slightly beyond the surface of the skin. Similarly, the external anchor can be configured to extend past the skin in order to contact the access port. The outer surface of the conduit 174 may form an ingrowth cuff with increased surface area that is positioned along the device where it contacts tissue and may facilitate tissue growth and seal reformation. Certain features including grooves or textures, can be applied to the ingrowth cuff to increase the maximum surface area of the cuff and increase tissue contact and growth. In some cases, specifically on or near the ingrowth cuff surrounding the access port, tissue ingrowth may not reduce the relative effectiveness or patency of the device. The advantage of tissue ingrowth at the delivery location 114 may be to improve seal or pleurodesis 112 formation. In addition, some growth surrounding the device may act as a flexible anchor and offer basic protection from impact or trauma.

FIG. 3 shows an exploded view of several components of at least one envisioned airway bypass device showing their engagement and interactions for delivery and deployment. Together, this view shows the combination of several components of the airway bypass device. The external anchor is shown interfacing and slideably receiving the access port and the fully deployed expandable structure 164. It is clear, from this illustration, that the diameter of the expandable structure 164 is significantly greater than the opening of the incision through the pleurae at the delivery location 114. A minimized incision length is typically preferred in minimally invasive applications. While the incision can be made in any direction between the ribs, FIG. 3 also shows at least one preferred incision formed parallel to the ribs to maximize the area made accessible by the incision.

In certain embodiment, it is envisioned that the external anchor 175 may be configured to engage the internal components of the device, specifically at the access port. In at least one exemplary embodiment, the external anchor may be configured to slideably engage with access port to secure the airway bypass device. As illustrated, the access port of the device may be dimensioned to receive the external anchor. As such, the access port may be configured with a greater diameter, such that the external anchor must be slideably received by the access port. Alternatively, the relative dimensions may dictate that the access port may be slideably received by the anchor. Finally, the internal and external portions may also connect via coupling, in which each device partially receives its respective complement. Regardless of dimensions, the ingrowth cuff of the airway bypass device should be positioned on the exterior of the combined port and anchor assembly to best contact the tissue of the chest wall. This contact may help to promote tissue ingrowth, seal formation and readily allow device security.

In some embodiments, it is envisioned that the interaction between the external anchor and the access port may cause the assembly to lock as the components slideably interact. In some embodiments, this interaction may result in a tactile lock that may be felt by the practitioner delivering the device. It is also envisioned, in at least one embodiment, that once the device is advanced a predetermined distance, the anchor and the access port may lock at that position allowing only for the continued advancement of the device. While it is envisioned that this locking feature may be reversed to reposition the device, as needed, the feature may also assist in applying the contact pressure needed to form the hermetic seal of the pleural cavity 134 from the atmosphere 50. Finally, the slideable interaction of the components may actuate the release of a delivery or installation tool used to deliver the access port 173 and expandable structure 164. This feature would facilitate the stepwise delivery of the device. Once the internal and external features securing the airway bypass device are fully assembled, a cap may secure to the exterior of the anchor.

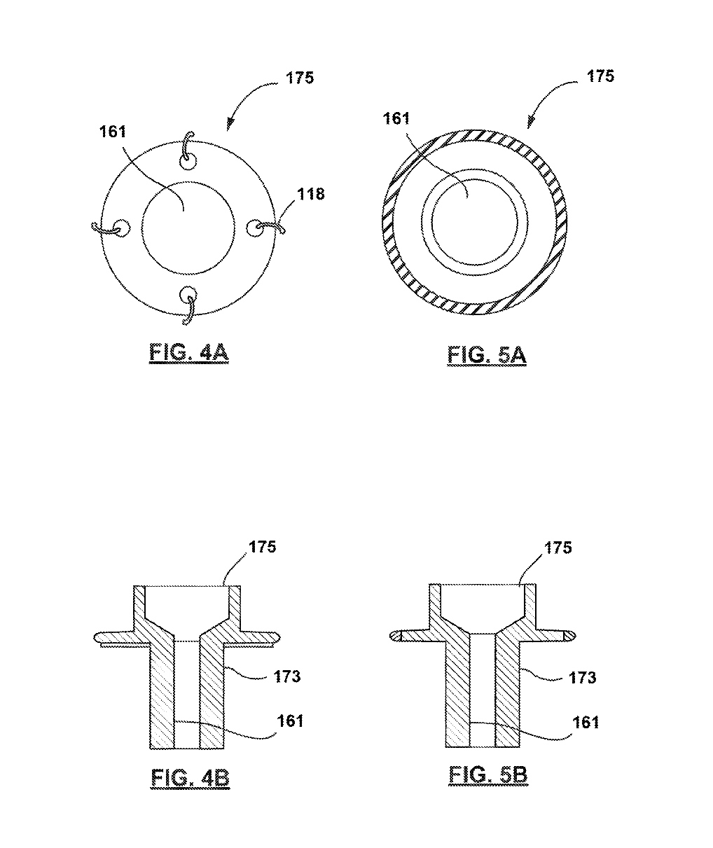

In some embodiments, external anchors, as illustrated in FIGS. 4A, 4B, 5A and 5B, may also be used to secure the device to the skin of the patient without drastically increasing local inflammation and irritation. FIG. 4A illustrates the use of sutures, one envisioned mechanism, for securing the external anchor to the skin. Once they are passed through the anchor, the sutures 118 may be tightened to secure bring the external anchor into contact with the surface of the skin. It is further envisioned that the fixation of the external anchor may be provided using several alternative or combined approaches. Alternative mechanisms, such as the dermal adhesive 146 may be used, when advantageous. Dermal adhesion can also be applied from the skin-face portion of the anchor, and can be used independently or in combination with other mechanisms, including suturing. As shown in FIGS. 5A and 5B, dermal adhesives 146 (without sutures 118 or other closure mechanisms) may provide the adherence to the skin to seal the access port.

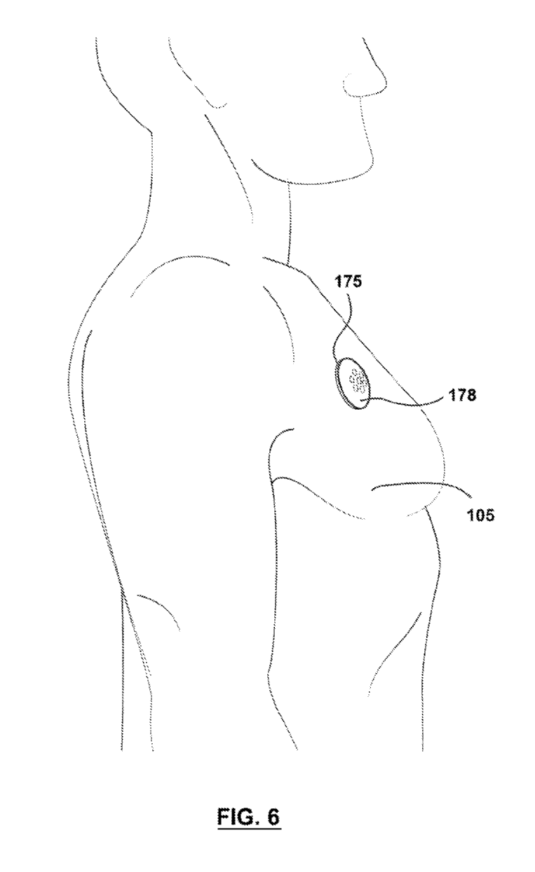

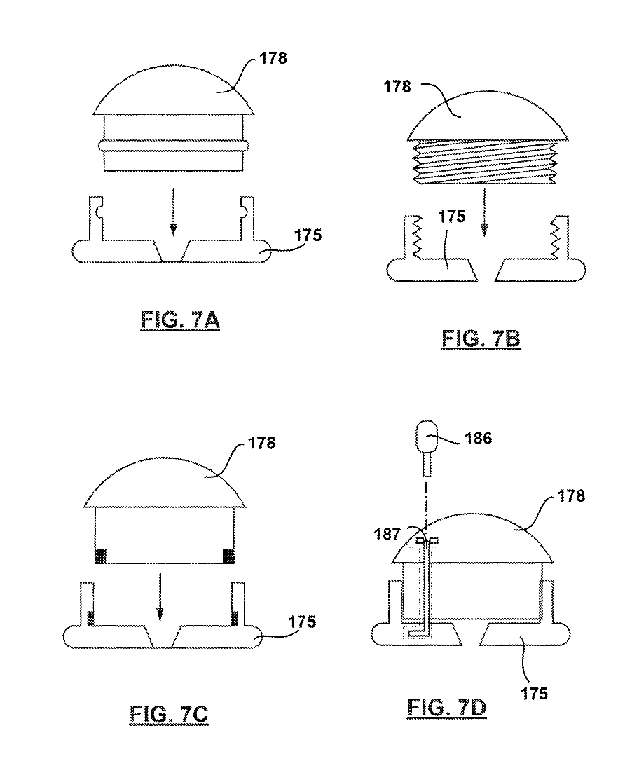

FIG. 6 shows an external anchor of a device mounted on the chest surface of a patient, with a tamper-safe or resistant locking mechanism or cap. Further embodiments of tamper-safe locking mechanisms or caps that may help to prevent unauthorized device access, while allowing continued fluid communication, are shown in FIG. 7A-7D. Locking mechanisms or caps may also be integrated with drug delivery or filter devices, as needed. FIG. 7A shows a cap 178 containing a mechanical detent 185 locking mechanism. The detent, which may take the form of a ridge, as illustrated, may also be configured with other shapes in some envisioned embodiments, including circular or other detents. It is further envisioned that the security provided by a locking mechanisms, such as a detent, might be sufficient to only reduce or deter unauthorized device access (i.e. the device would still be accessible under emergency conditions). In other instances, it is envisioned that separate device, one available to the practitioner, may be used to release the cap from the anchor to allow access to the device. Thus, the cap may attach to the end of the conduit by at least one of a lock, ring engaging a lip, an O-ring seal and a threaded male and female screw arrangement.

It is also envisioned that other locking mechanisms may be used to secure the cap to the corresponding anchor. FIG. 7B shows a screw-in locking mechanism, which would be tightened sufficiently to prevent or deter unauthorized device access. FIG. 7C shows a magnetic locking mechanism, in which magnetic portions of the cap and anchor components (shown as darkened portions) may supply an electromagnetic force sufficient to make unauthorized device access difficult. It is envisioned that the magnetic mechanism in at least the cap, or anchor may be disabled to allow for device access. It is also envisioned that some embodiments or combinations may employ locking mechanisms that may be accessed with physical keys, as seen in FIG. 7D. A specifically configured key 186 may be used to access a key hole 187 to release the cap from the anchor to allow access to the device and internal lung. Locks with electronic keys, acousto-magnetic systems, magnetics system, RF systems, and combinations thereof are also envisioned.

In addition to rigid, tamper-safe mechanisms or caps, it is envisioned in at least one embodiment, that disposable or replaceable external cover may be used to deter unauthorized device access. As shown in FIG. 8, it is envisioned that a sponge-like component with porous structures may be positioned under or in place of the cover or cap and may act to prevent or deter unauthorized device access, while permitting the continued fluid connection between the internal lung and the atmosphere. In certain instances, the pore size could range from 0.22 um up to 3 mm and 0.5 mm to 2 mm. Further, as shown in FIG. 8, the dimensions of the structure may be determined relative to the external anchor, with distance “d” representing the diameter of the sponge cap to fit on the external anchor, while distance “D” is envisioned to be equal to or larger than the outer diameter of the external anchor.

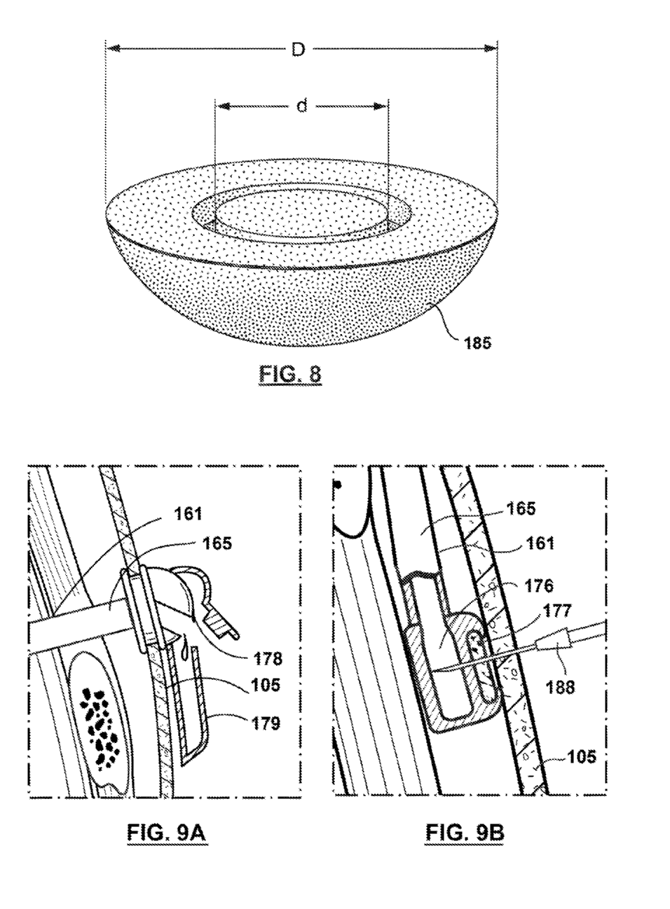

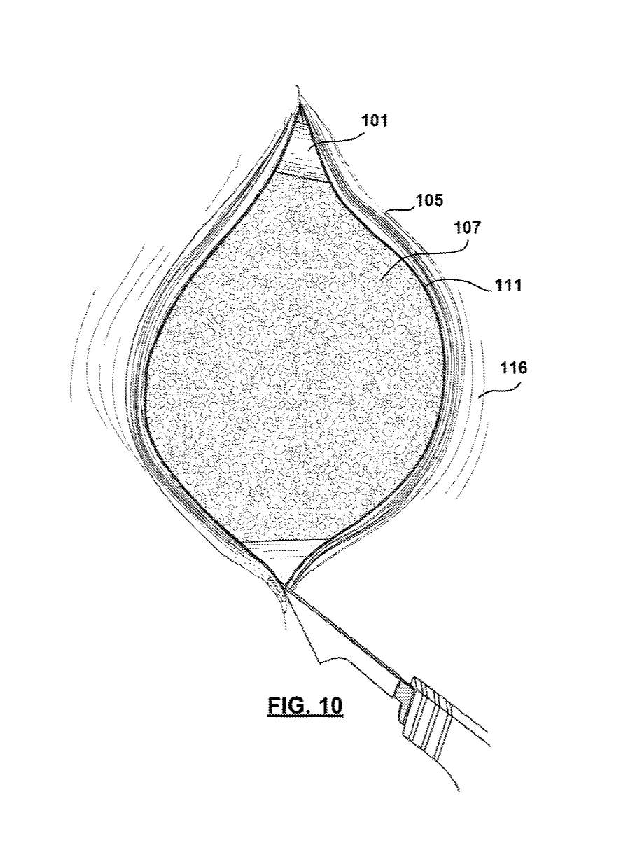

Further components, as shown in FIG. 9A, such as vent caps, valves , filters, connectors, fluid traps, ingrowth cuff located at or near the external portion of the device may then be fastened, inserted or otherwise secured to the device. It is envisioned that the a cap 178 covering the lumen 169 serves an additional safety function by restricting or partially restricting user access to the device. As shown, it is envisioned that a safety cap 178 might be located upstream of patient serviceable parts including at least the fluid trap 179 and filter 185 and may contain a locking mechanism to prevent unauthorized access to the internal device or lumen 169. As such, in a preferred embodiment, the device may be configured to accommodate patient adjustment without allowing unsafe access or device removal. In one envisioned embodiment, the cap 178 contains a magnetically locking mechanism. Other envisioned embodiments include locks with physical keys, locks with electronic keys, acousto-magnetic systems, magnetics system, RF systems, and combinations thereof.

An external cap 178 may limit device access, but may also provide for a mechanism to connect to pathways for diagnostic, interventional or treatment devices. Interventional procedures ranging from tissue biopsy to device cleaning may be performed through the device plug or septum 177 under the external cap 178. Further device related procedures may include in situ diagnosis, RF ablation, laser treatment, or argon plasma coagulation (APC). Finally, the cap 178 could be adapted for use in the delivery of localized treatments for non-COPD related disease, if using proper tools and technique. Drug delivery with an adapted cap may provide a pathway for quick and direct drug delivery to diseased tissue in or even beyond the lung.

In addition to limiting device access, it is envisioned that external features including a filter may form a selectively permeable membrane that may exclude the passage of certain material into or out of the device. The filter may reside long a portion of the conduit, or within the external anchor or cap assembly. In at least one embodiment, the cap assembly comprises the filter element, which allows the filter to be replaced once the 178 is removed. In at least one aspect, selective passage may be used to maintain a level of sterility within the implanted components of the device and/or foreign material from entering device. Multiple layers of filtration may be combined to narrow the selectivity of this membrane, as needed.

It is envisioned in at least one embodiment that the filter may reside within the access port 173 and may help to form a barrier between the external environment and the internal lung. Maintaining the partial isolation of the body cavity may help to reduce the risk of environmental contamination and infection, and maintain body and device sterility. A filter may prevent the passage of material based on the size of the particulates. For example, a filter may reduce the passage of particulate size less than 5 microns, or less than 2.5 micron. As such dust and other common environment particulates may be kept from entering body. Alternatively, a filter may be dimensioned to restrict passage of contaminants larger than 0.22 microns in size to exclude microbes, but allow for the free flow of gas.

The device filter may also provide selective passage to water through size or chemical (hydrophobicity) constraints. A hydrophobic filter may prevent body fluid such as sweat and water from reaching body cavity. Specifically, hydrophobic filters of less than 0.22 um could effectively perform a combination of many of the above functions. Filters of specific dimensions, including larger pore filters may be desired to achieve more of the above functions when device and body sterility is not critical to patient heath.

Alternatively, FIG. 9B shows an alternative configuration to the end of the conduit 161 of the airway bypass device 150. Instead of passing through to the external atmosphere 50, FIG. 9B illustrates the bypass device connected to a subcutaneous port, wherein the subcutaneous port with a fluid reservoir is configured to allow selective access to the conduit connected to the bypass device. It is envisioned that the expandable structure 164 of the device is connected to a subcutaneous structure with reservoir inaccessible from the exterior except through a specialized cover. To be fully implantable underneath the skin, the reservoir is envisioned to be housed within a rigid subcutaneous reservoir 176, fully constructed of biocompatible materials. It is envisioned that the entire subcutaneous reservoir 176 may be embedded under the patient skin and may be dome-shaped and sealed from the environment by an elastic plug 177. When the plug 177 is configured to be penetrated to provide access to the reservoir, a self-sealing septum may be advantageous. Isolation from the exterior environment may aid in reducing the chances of contamination or unauthorized access to the device, device conduit, or the patient pleura. The subcutaneous reservoir 176 may be substantially flat facing the patient body and comprises an outward facing septum 177, preventing reservoir access. The reservoir is fluidly connected to the device conduit of the device via an outlet aperture on one side of the subcutaneous reservoir 176. As needed, a user or practitioner may choose to establish fluid communication between the reservoir 176 and the external environment by physically traversing the seal. The connection may be made with the aid of a specifically configured septum-safe device.

It is further envisioned that the fluid reservoir may be sealed from external communication by a flexible septum 177 formed from a resiliently deformable material covering the fluid reservoir and the outward facing portion of the subcutaneous reservoir 176. The septum 177 may be configured of any suitable biocompatible material such as, for example, silicone. A silicone septum 177 is generally “self-healing” in that it is able to substantially return to its original shape and seal after one or more non-coring punctures. This self-healing feature is generally achieved by applying a compression to the septum 177, as configured over the reservoir below. This force may be achieved by sandwiching the septum 177 pressed between two pieces of the body during device delivery, such that compression is achieved and maintained after device implantation. Temporary applications may benefit further from device implantation within tissue, as tissue growth can produce a secondary seal in response to perforations, in addition to the elastic seal formed by the septum 177 material. As such, it is envisioned that numerous perforations may be made before the septum 177 forms a seal ineffective for use.

Some envisioned septum-safe devices, also called Huber needles, may include needles configured with deflected points or designed with a non-coring point to eliminate the potential of “coring”, in which a portion of the elastic self-sealing septum 177 or port is removed entirely from the seal. The directional configuration of the needle 188 point prevents damage to the septum 177, while still allowing for fluid communication between the reservoir and the exterior. The configuration further prevents the needle 188 interior from being occluded by a potential septum 177 core. Upon removal of the deflected needle 188, the septum 177 automatically reforms a seal from the exterior environment.

It is further envisioned that in some embodiments, the subcutaneous reservoir may be anchored to the patient below the surface of the skin to avoid the external exposure of components. Because the device may be embedded or anchored beneath the skin, no superficial active site maintenance is envisioned and the need for long-term maintenance may be reduced. The absence of external components allows the port and device to be unobtrusive in the daily life of patients. Such applications place fewer restrictions on patients and may afford users and practitioners the potential for immediate drug delivery to the distal portion of the lung and access to a bypass device on an as-needed basis, all without negatively impacting overall patient quality of life.

In at least one method of use, it is envisioned that the fluid reservoir is accessed on a regular maintenance schedule. Access may be made with the use of a needle 188 with a deflected point or non-coring point. Such a needle 188 may be inserted through the plug or septum 177 to reach the reservoir 176 below. The excess gas in the reservoir or pleura can be extracted or vented through the needle 188 into the external environment, and the needle 188 carefully removed to allow the elastic seal to reform. In another method of use, it is envisioned that access to the bypass device may only be needed intermittently and can be made on an as-needed basis. One advantageous application may be in patients where access to the bypass device is only needed infrequently, and can be self-initiated when the patient experiences usually severe symptoms such as shortness of breath, difficulty breathing, wheezing, etc. Finally, in both scheduled and intermittent applications, drug delivery to even the most distal bullae of the lung is feasible with the envisioned application. In addition, the shortened delivery route reduces drug diffusion and increases access to locations conventionally regarded as the least accessible areas of the lung.

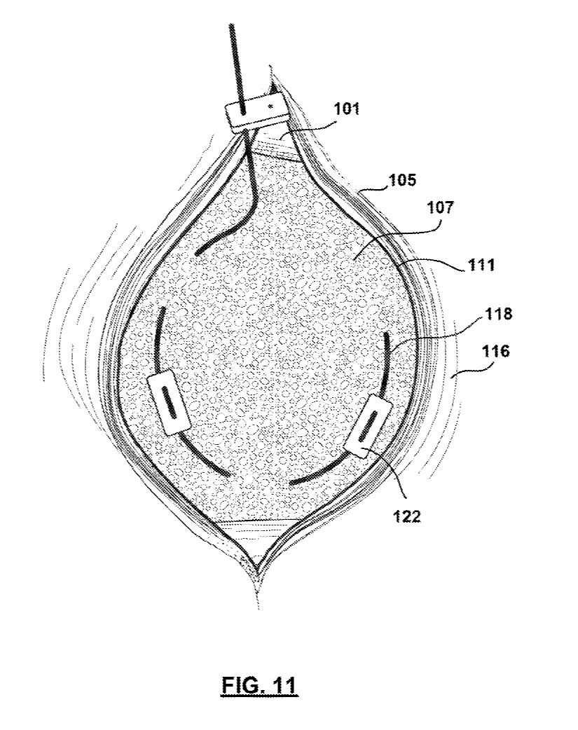

It is envisioned that at least one exemplary minimally invasive surgical technique may generally comprise the steps that are described and detailed herein. These steps may be used to secure the lung and mitigate the risk of complications from pneumothorax. FIGS. 10-16 shows at least one incision and suturing technique that may be used to minimize the risk during device delivery. Such a technique could be used in a minimally invasive delivery of an airway bypass device.

In FIG. 10, the intercostal space between two ribs is selected for device delivery. A typical incision 116 may be made within the second or third intercostal space, but any location with access to the lung may be selected, as needed. Avoiding restrictive structures such as the ribs 101 or heathy areas of lung parenchyma 106 may narrow the candidate delivery locations 114. While the incision is illustrated vertically, it may be preferred that an incision be made parallel to the ribs (i.e. horizontally or substantially horizontally) to maximize the area made accessible by the incision, while avoiding the rib entirely. The delivery location 114 containing the first incision should be made at a depth sufficient to fully pass completely through the dermal layers, and muscles of the chest wall. However, only the chest is perforated revealing the outer layer of the lung. Care is taken not to damage the underlying pleurae 107, 108, as to prevent the de-pressurization of the pleural cavity. Sutures 118 are then put in place through the pleurae 107, 108 and used to hold both layers against the chest wall before a second incision through the layers is made. The second incision will subsequently expose the pleural space to the external atmosphere, which allows for device delivery. However it is envisioned that the sutures 118 are sufficient to bias the lung to prevent complications from arising due to pneumothorax.

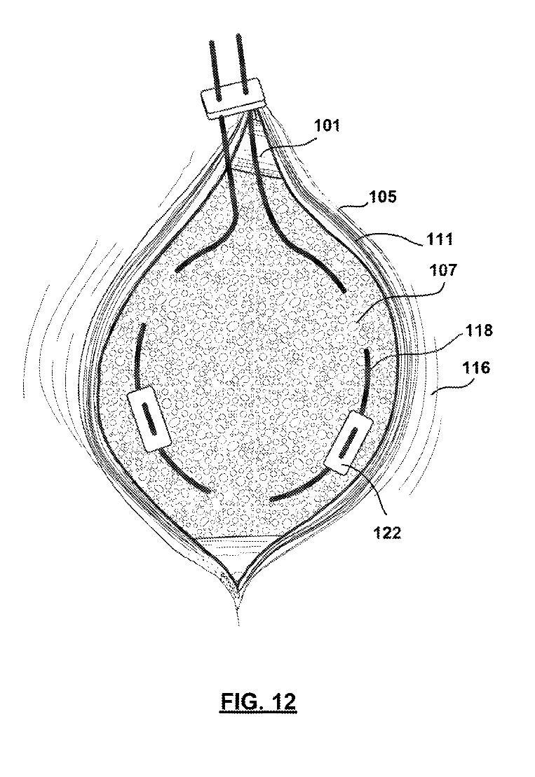

FIGS. 11 and 12 show the use of pledgets 122 in addition to a mattress suture through the pleurae 107, 108 of the lung. To prevent the tearing of the pleurae 107, 108, pledgets 122 may be used to increase the surface area grasped by the suture. The increase in surface area would allow for the distribution of the force of the thin sutures 118 across this larger surface area. It is envisioned that the larger surface area provided by the pledgets 122 may help to prevent the so-called “cheese wire” effect, in which narrow structures (e.g. sutures, wires, etc.) pass partially or completely through tissue as the biasing force from those narrow structure exceeds the ability of the tissue to hold said structure.

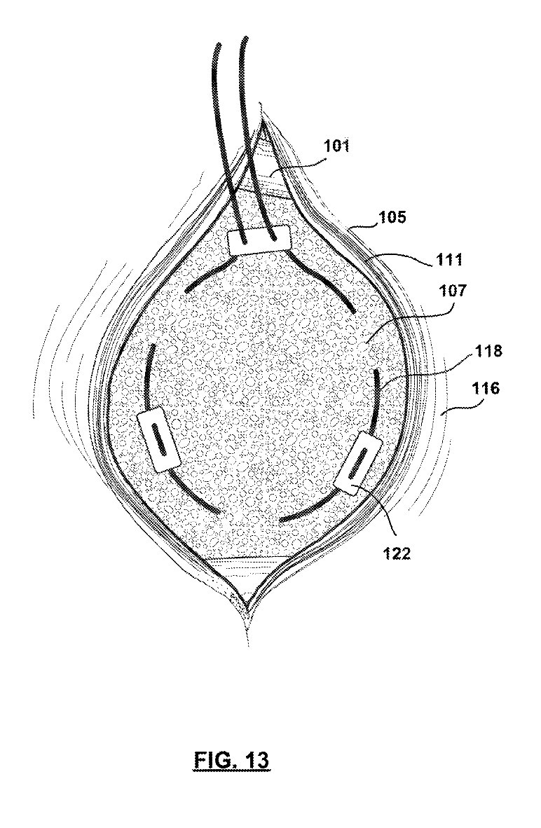

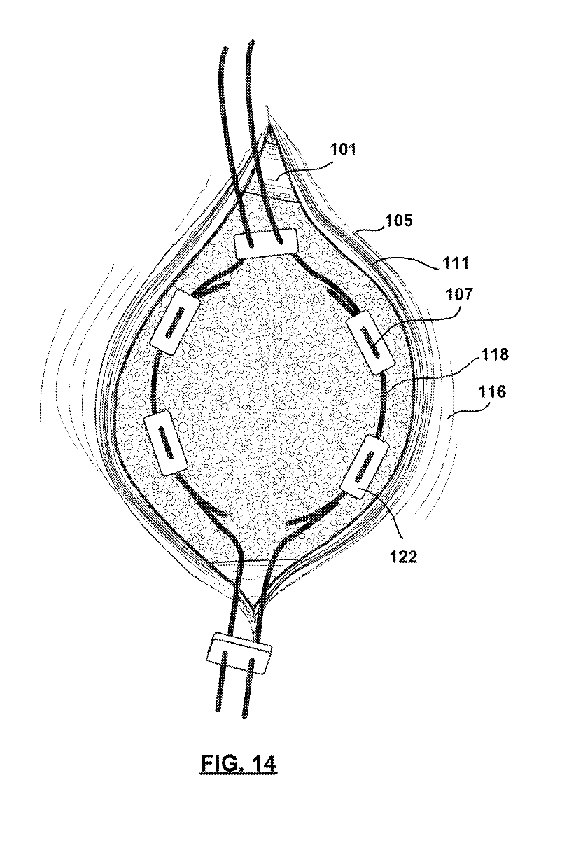

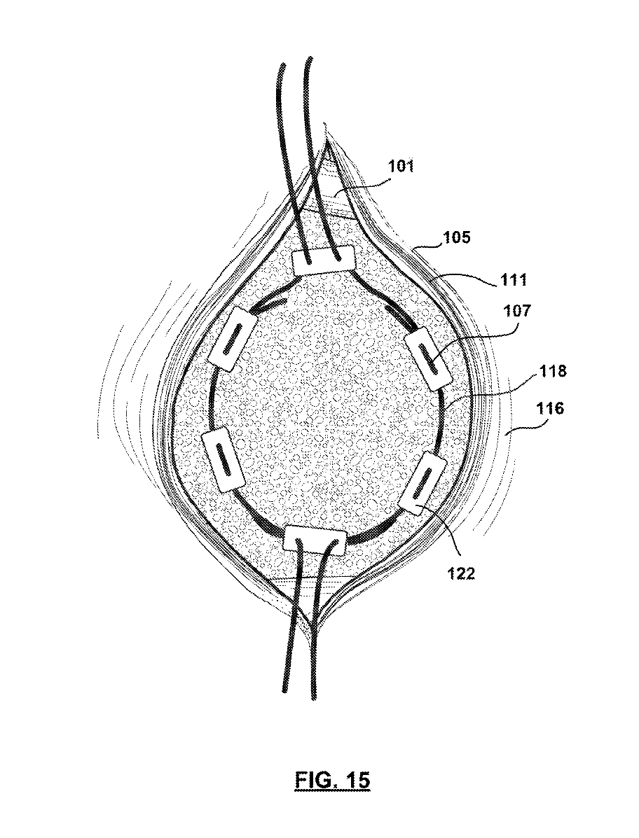

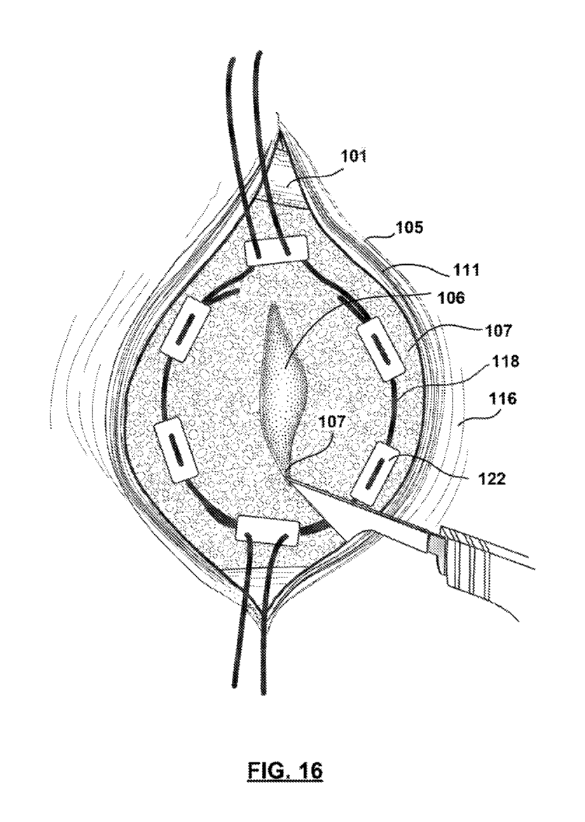

A second running (e.g. purse string, mattress, etc.) suture may be added opposite to the first, as shown in FIGS. 13-14 to further distribute the force of the sutures 118 against the pleurae. Alternative suturing methods or the use of additional running sutures that are not shown may also be employed. As shown, a second suture could be positioned primarily around the exterior of the pleurae in the areas not covered by the first suture. In certain delivery methods, one or more sutures 118 may be deployed to form a full ring around the section of pleurae selected for device delivery, as shown in FIG. 15. FIG. 16 shows the completed preparation with the sutures 118 knotted and the second incision made to allow for the next steps of device delivery. As illustrated, the second incision is made through the parietal pleura 108 and the visceral pleura 107, exposing the lung parenchyma 106. The device may then be delivery through this second incision into the internal lung.

FIGS. 17-34 show a sequence of cross-sectional side views that illustrate at least one envisioned method for installing (e.g. delivering, deploying, etc.) a lung airway bypass device 150. Beginning with FIG. 17, the sequence illustrates one envisioned method of lung biasing, creation of one or more incisions and device delivery. In FIG. 17, the chest is shown prepared for device delivery, generally following the steps shown in FIGS. 10-16, with sutures 118 applied to the pleurae of the lung to prevent the lung 100 and the associated lung parenchyma 106 from migrating away from the chest wall 111 during device delivery. A guide wire 124 is oriented toward the patient, in the direction of the skin surface. While the body is shown in a vertical orientation, it is envisioned that it may be advantageous to maintain the body in a supine position during device delivery. Alternative orientations may also be selected based on the target location for device delivery or as needed to facilitate access to the delivery location 114 by the practitioner performing the device delivery.

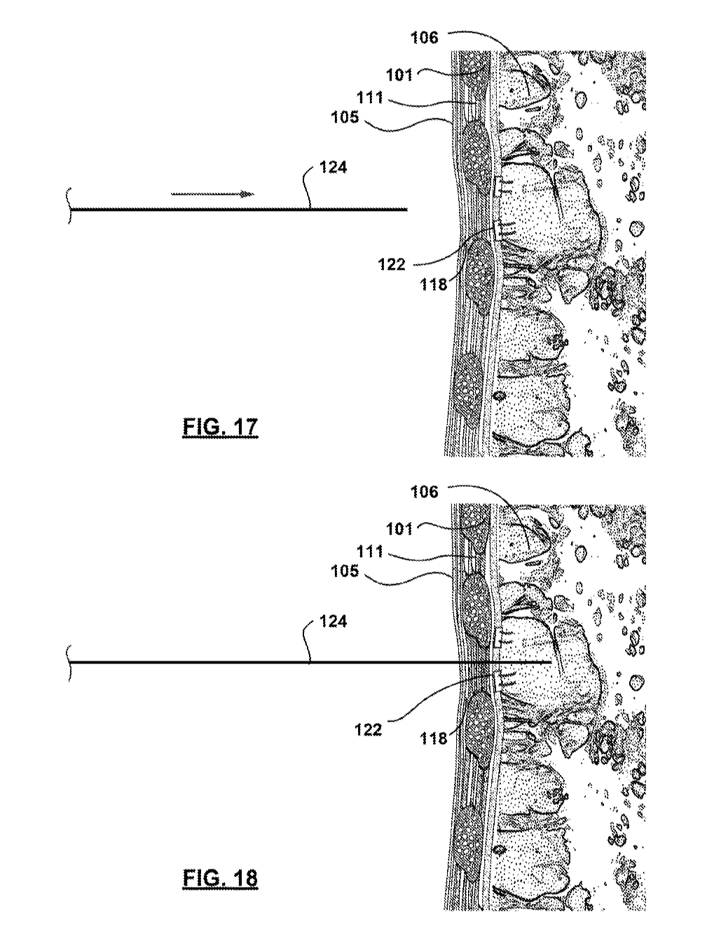

As shown in FIG. 18, the guide wire 124 may be advanced through the chest at the delivery location 114. If one or more incisions has been made, the guide wire 124 may be advanced through the first and second incision 116, 136 at the delivery site. When only a first incision has been made through the chest wall, the narrow guide wire may be passed through the first incision and then through pleurae 107, 108. A needle with a narrow profile capable of entering the lung and dimensioned to accommodate the guide wire 124 may also provide an alternative method of guide wire delivery. A visual depth indicator present on or along the surface of the guide wire 124 may help in forming an estimate of depth to supplement any direct visualization used during device delivery.

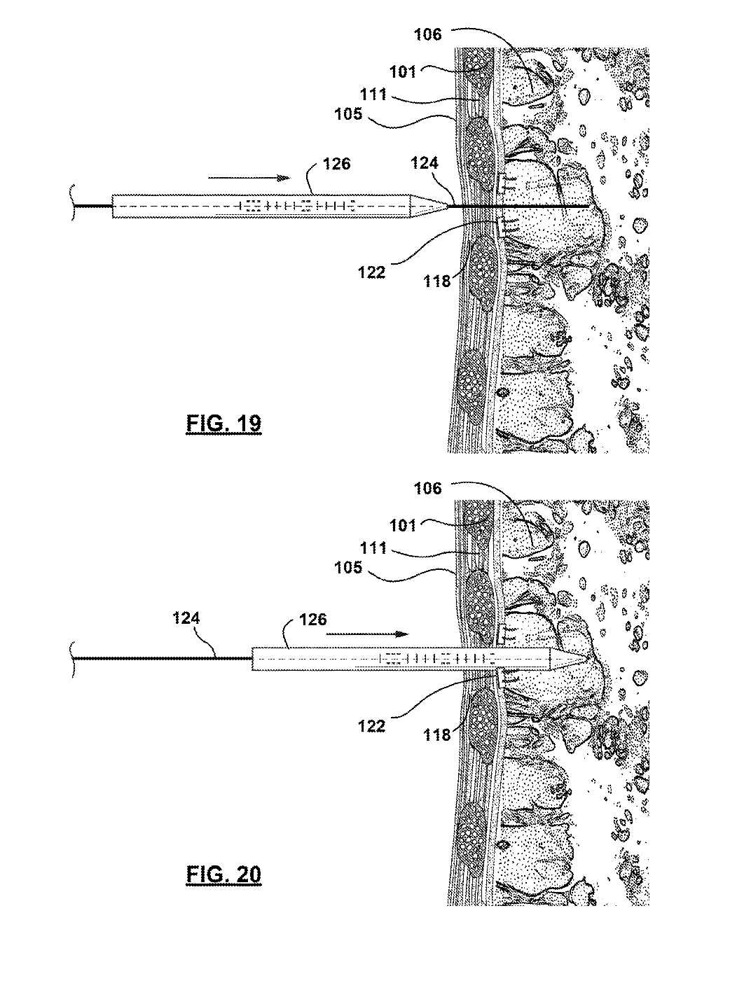

Once the guide wire 124 is in the place, one or more tubular dilator 126 may be advanced, as illustrated in FIGS. 19 and 20 along the guide wire 124 into the lung 100. When additional dilators are used in addition to the first, it may be preferred that the diameters of any subsequently deployed dilators is gradually increased. The deployment of additional dilators or sheaths follows the steps illustrated in FIGS. 19-22, wherein the larger-diameter dilators or sheaths are advanced over the current components. In the case of gradually expanding dilators, the small-diameter dilator may be removed upon the delivery of a larger dilator. These steps may be repeated until the diameter of the dilator or delivery location 114 is sufficient to accommodate device delivery. As such, this method of delivery may provide for the safe delivery of airway bypass devices of various sizes using a safe and standardized approach. Once again, depth indicators (e.g. markings, notches, etc.) may allow a practitioner to quickly estimate depth and confirm dilator and device location to supplement or in place of direct visualization.

In at least one embodiment, dilators 126 may be formed of a rigid or semi-rigid material. A semi-rigid dilator may be advantageous in reducing local inflammation and trauma. In addition, flexibility may be needed to avoid certain areas within the lung parenchyma 106, that may be previous identified using traditional visualization methods. The dilator may comprise a conical tapering tip to facilitate advancement into the lung. In some instances, the diameter at the tip of a dilator 126 may only be slightly wider than the guide wire 124 diameter. A tapering conical shape facilitates the use of the dilator 126 in penetrating the chest wall 111 and pleurae 107, 108, as needed to follow the path of the guide wire 124. In addition, the shape may effectively reduce the local trauma to tissue by reducing the amount of tissue displaced by the insertion of subsequently larger dilator 126 diameters.

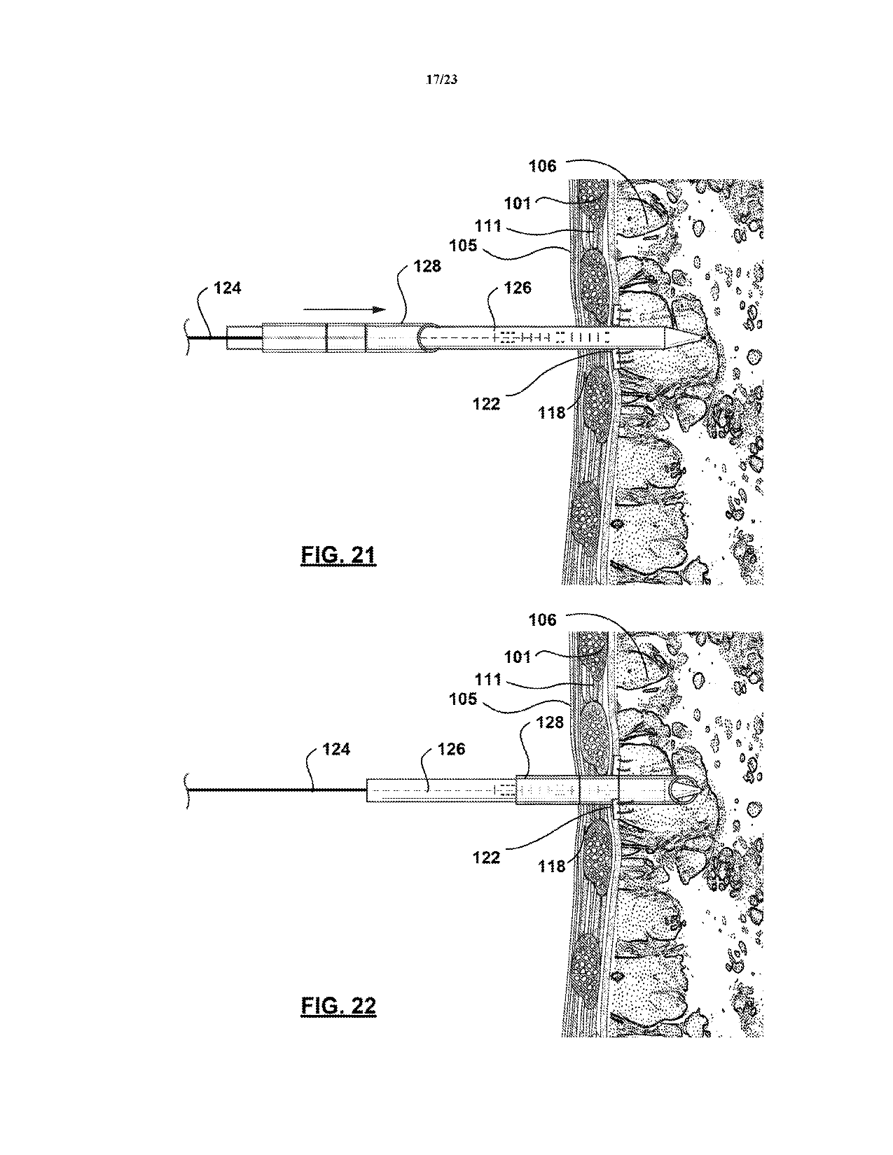

In FIGS. 21 and 22, a final dilator sheath 128 may also be inserted over the guide wire 124 and dilator assembly. The dilator sheath 128 is dimensioned similarly to the dilator 126, but is generally envisioned to be dimensioned with a single uniform diameter. In some instances, the dilator sheath 128 may also be formed of a flexible or semi-rigid material. In other instances, it is preferred that the dilator sheath 128 be formed of a rigid, durable material to ensure the diameter of the sheath will accommodate device delivery. In some instances, the tip of the sheath 128 may be configured with a tapered or beveled shape to facilitate advancement into the lung. Once the dilator sheath 128 is fully advanced, as shown in FIG. 22, any remaining dilators 126 may be removed, leaving the sheath 128 and guide wire 124 assembly of FIG. 23.

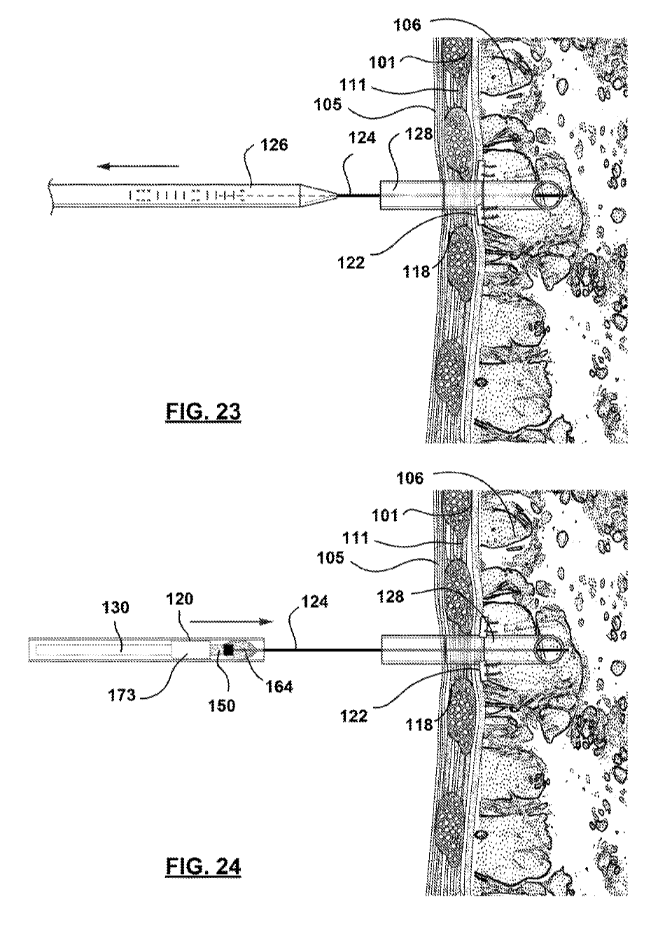

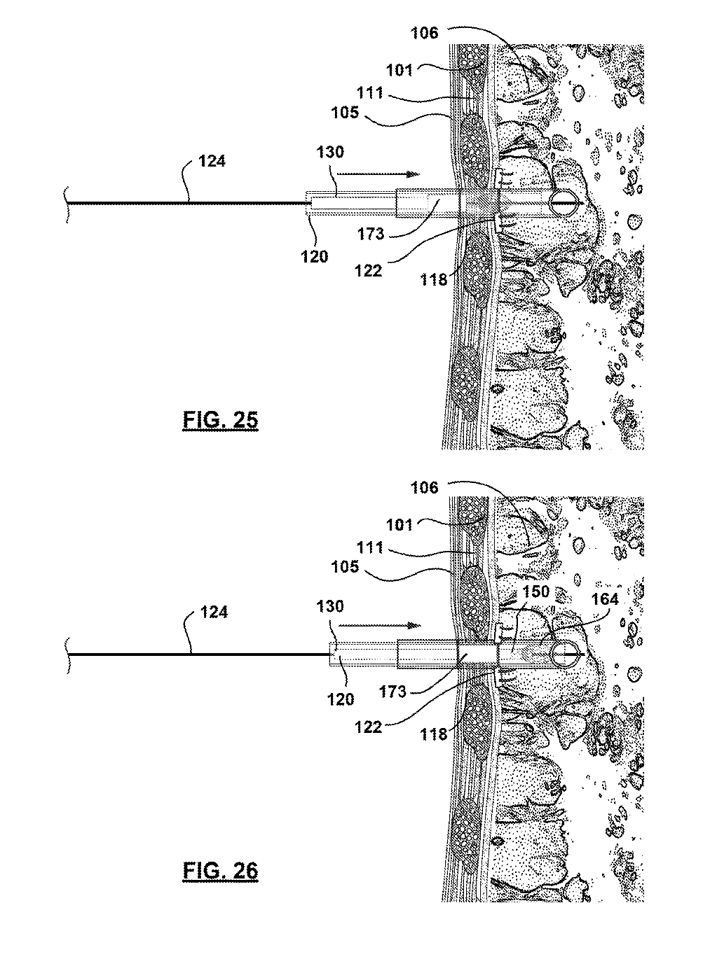

Once the dilator 126 is fully retracted, the airway bypass device 150, which may be held in an unexpanded, compressed form, may be advanced along the guide wire 124 for delivery. In at least one embodiment, the airway bypass device 150 is compressed using a delivery sheath 120 that surrounds any expandable structures 164 of the airway bypass device 150. This compression temporarily reduces the profile of the airway bypass device 150, and helps to ensure that the diameter of the device is sufficiently reduced to allow passage through the dilator sheath 128. FIG. 24 shows the advancement of the device towards the delivery location 114 and into the dilator sheath 128. FIG. 25 further illustrates the relative dimensions of the sheath and device envisioned to facilitate unobstructed passage through the chest wall and pleurae. Finally, FIG. 26 shows the airway bypass device 150 fully advanced at the delivery location 114. FIG. 26 illustrates the ideal position envisioned for device delivery with the access port of the airway bypass device 150 spanning the chest across the skin, chest wall and both pleurae 107, 108. It is further envisioned that the ingrown cuff on the outer surface of the conduit 174 may be deployed on the airway bypass device may preferably surround the access port at the locations where contact is made with the tissue of the chest wall.

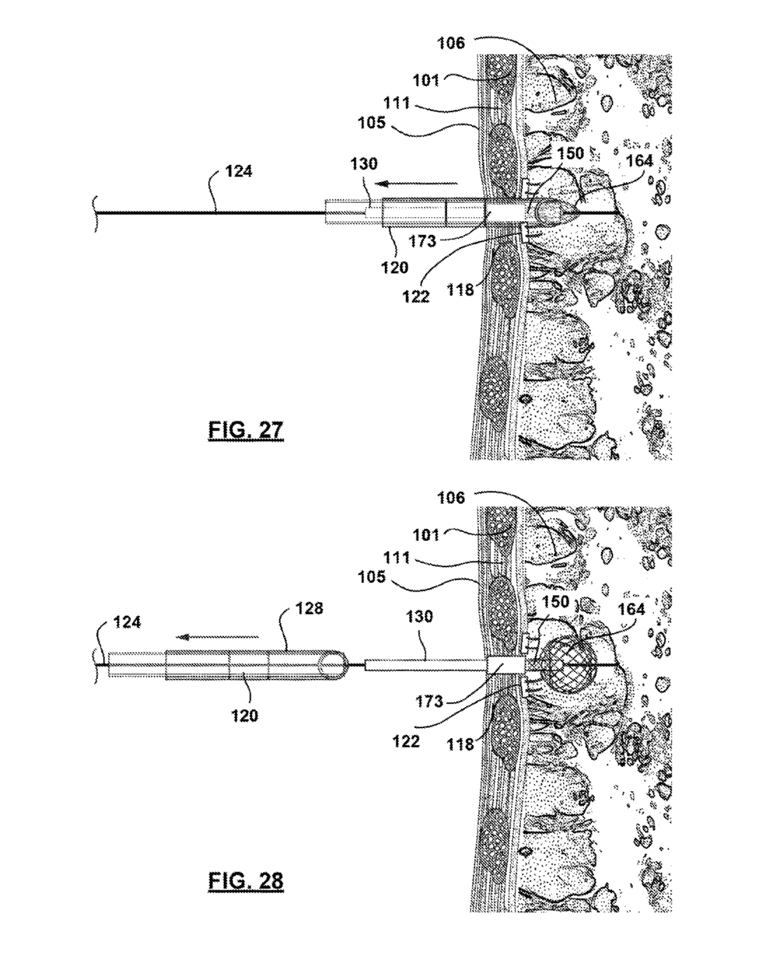

FIGS. 27 and 28 illustrate the steps envisioned for device deployment. Following device delivery (i.e. positioning of the airway bypass device 150 within the lung) the dilator sheath 128, and other remaining delivery mechanisms, may be retracted from the bypass device. FIG. 27 shows the expandable structure 164 of the device partially freed from the dilator sheath 128 and shows the dilator sheath 128 completely retracted from the delivery location 114 in FIG. 28. As shown, the expandable structure 164 may automatically expand in size due to its shape-memory construction. It is envisioned that the guide wire 124 and an installation (i.e. delivery) tool 130 may remain once the sheath is fully retracted. In another embodiment of the placement procedure, the dilator sheath may be inserted into the parenchyma of the lung 106 without the use of the needle, guide wire, sheath as previously described. Instead, the sheath and device could be inserted through a small incision made in the center of the purse-string sutures and deployed then subsequently follow the delivery method described. By not utilizing the dilators, the procedure time would be reduced, thereby helping to minimize complications (e.g. pneumothorax) resulting from the use of multiple device and exchanges through the pleura.

Finally, FIGS. 29-34 show the additional delivery and installation of an external anchor 175. Although an external anchor 175 is optional for use with an airway bypass device 150, anchoring may be used to help seal and secure the airway bypass device 150. It is envisioned in some embodiments that an external end of the device may be configured to remain external or partially external to the patient. An external anchor 175 may be secured to the outer chest wall, and comprise additional skin mounted anchors, such as an inward facing surface with an adhesive configured to be affixed directly to the patient skin or apertures, holes, or slots that can be filled with silicone or provide a surface for suturing.

Sutures 118 or other fixation mechanisms may attach the external anchor 175 to the skin or internal tissue of the patient. Although envisioned in some aspects, directly suturing (e.g. purse string or mattress suturing) the external anchor 175 to the skin or tissue may not be necessary. Instead, in some embodiments, the skin may be closed over the access port 173 of the external anchor 175 by sutures 118 or other closure mechanism. Tightening of the opening of the skin at the delivery location 114 may be sufficient to secure the device. After the access port 173 is inserted, the skin at the perimeter of the opening may be pulled together at the access port 173 to close any remaining opening to tissue.

Temporary or permanent fixation may be preferred at several locations and stages during device delivery. It is envisioned that various other methods of closure, skin sealing, and tissue growth may be used to form a secure seal around ingrown cuff of the access port 173 (corresponding to the conduit 174) of the airway bypass device 150. The outer surface, e.g., ingrown cuff, of the access port 173 may have a surface or be coated with a material to promote attachment of skin and tissue to the outer surface of the access port 173. From securing the access port 173 of the device, to contacting tissue to the ingrowth cuff, fixation of the device serves a critical aspect for both sealing and securing the device.

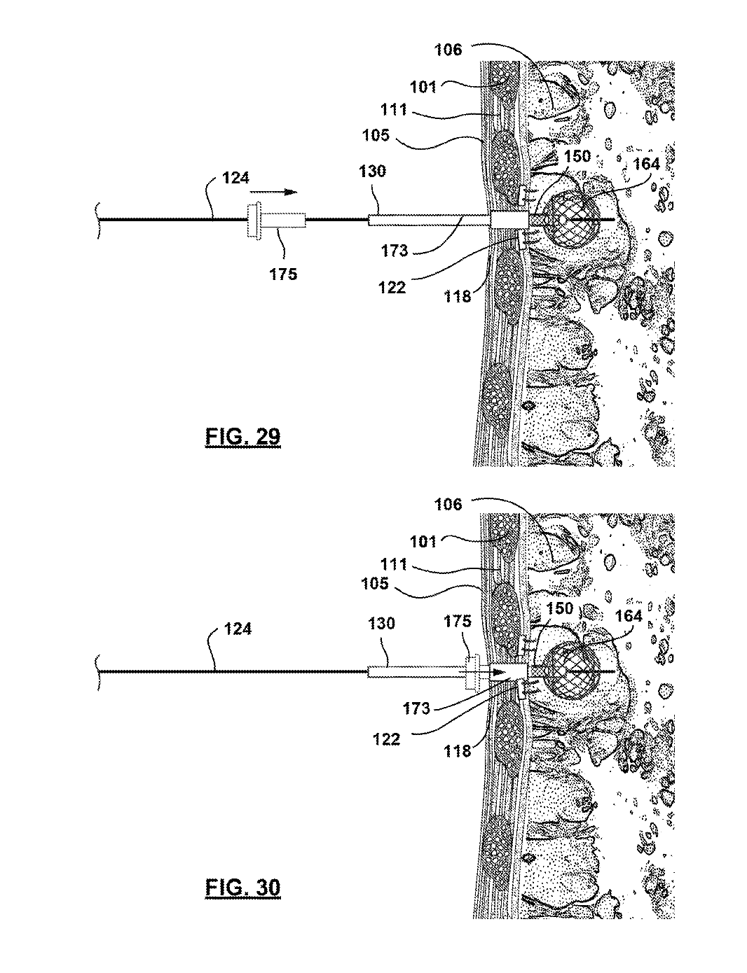

If sutures 118 or other fixation mechanisms are used in facilitate the delivery of the access port and bypass device, these sutures 118 may be tightened before the delivery of the external anchor 175. FIG. 30 shows the insertion of an external anchor 175 into the access port 173 of a device. The dimension of the external anchor and the access port 173 of the device may be within a range such that their relative sizes vary. As such, the external anchor 175 and access port 173 may each be slightly larger in diameter than the other. However, in each of these cases, it is envisioned that the interaction between the external anchor 175 and the access port 173 may cause the assembly to lock as the external components slideably interact. The interaction may result in a tactile lock that may be felt during device delivery, offering confirmation of anchor 175 advancement. It is also envisioned that once the device is advanced a certain distance, the anchor 175 may lock at that forward position allowing only for the continued advancement of the device. While the locking feature may be reversed the contact pressure formed by the coupling of the external anchor 175 into the access port 173 may help to reform the hermetic seal of the pleural cavity 134 from the atmosphere 50.

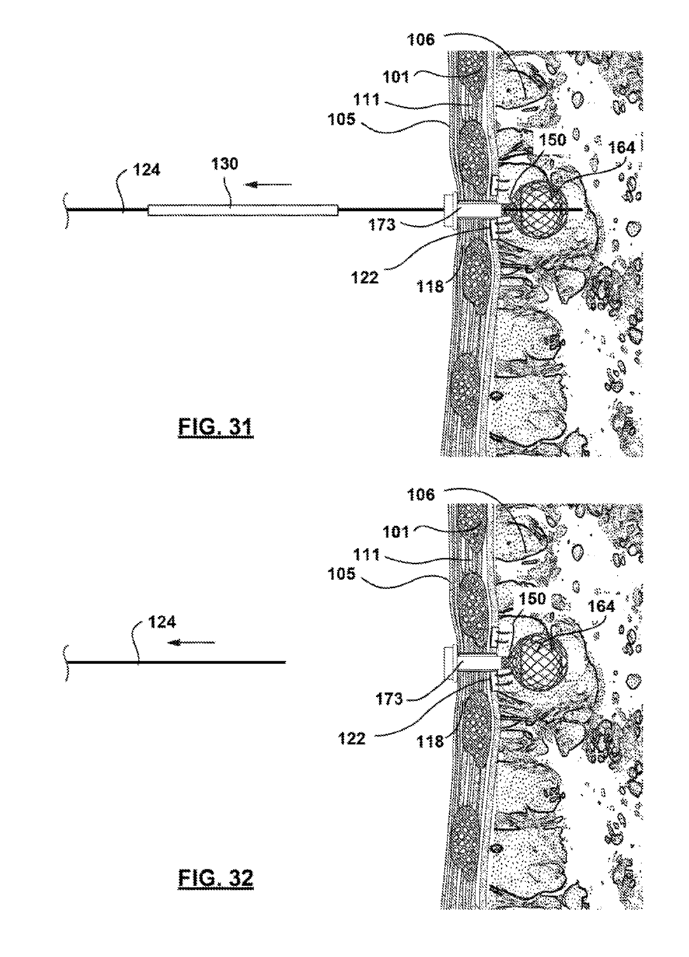

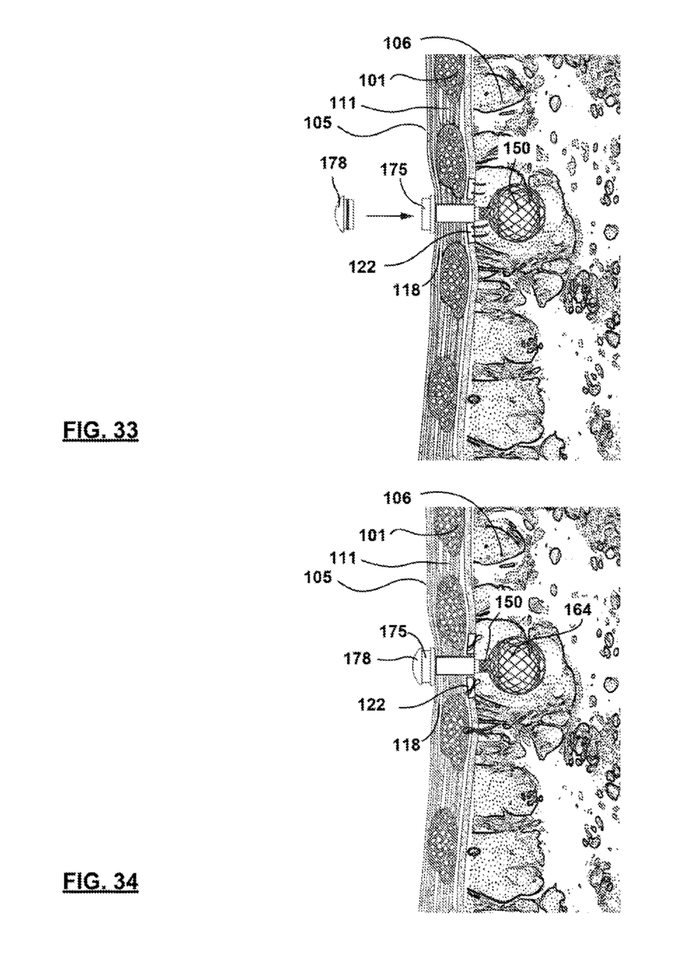

FIG. 31 shows the subsequent removal of devices associated with device delivery. The interaction of the anchor 175 components may actuate the release of a delivery or installation tool used to deliver the access port 173 and expandable structure 164. This release mechanism may help to ensure that the device is properly delivered and positioned before allowing the positioning of additional components. Finally, FIG. 32 shows the removal of yet another delivery mechanism, the guide wire 124. FIG. 33 shows the installation of a tamper-resistant external cap and FIG. 34 shows at least one envisioned airway bypass device 150 fully installed. It is envisioned that delivery of the each of these components (e.g. external anchor 175, cap, etc.) may be used to actuate mechanisms that tighten or release one or more of the devices associated with device delivery and deployment, ensuring the stepwise delivery of the device.

It is envisioned in certain aspects that it may be advantageous to shorten the time required between the creation of a first and second incision and closure associated with the delivery of the device. Therefore, it may be desired, in some aspects, to further facilitate device delivery by combining the performance of the incisions and closure step into a single step.

In one or more of the embodiments described, it may be advantageous to use a tissue glue (e.g. lung sealant, a soft tissue glue) injected between the device, internal flange or anchor and the internal surface of the chest wall to enable adhesion at a relatively low contact pressure. Tissue glue may also be used to provide additional adherence in a port or internal flange. In one embodiment, glue may help to maintain a seal even if the compressive pressure applied by the internal flange on the tissue is relieved.

Optionally, air-venting, vacuum or suction may be applied to the expiration or external end of the device. In addition to these features, the device may be configured to connect to other components external to the patient. For example, the device may further comprise one or more external ends in the form of fitting, such as a luer adaptor or a clamp adaptor. This connector may be used to connect instruments used by a physician to perform cleaning, diagnostic, drug administration, or various other functions.

It is important to note that, while the order or arrangement of the components might be interchangeable, there may be an arrangement or multiple arrangements that are advantaged, as described. While no particular order to the plug or septum 177 fluid trap 179, cap 178, fitting 182, or filter 185 is preferred, in at least one embodiment the filter is configured nearest to the air intake component 165 to prevent or reduce filter occlusion. In this or in other embodiments, the fluid trap 179 and cap 178 may be place on the exterior of the patient.

The device may be configured with radiopaque areas to allow imaging technology to assist in assessing if the device is implanted satisfactorily. Imaging may also be used during and following the steps of implanting the device to facilitate the proper placement of the device. Imaging technology such as x-ray or fluoroscopy may be used to image radiopaque markers placed on the device, for example on the expandable structure 164 or access port 173.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s).

In this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. An airway bypass device configured to form an air bypass passage to a diseased lung comprising:
an expandable a permeable structure configured to be embedded in lung tissue and defining within the permeable structure an air capture chamber, wherein the permeable structure includes at least one opening through which passes air from the lung tissue into the air capture chamber;
a first conduit having a first end which is open to the permeable structure and an opposite end configured to couple to a second conduit, wherein the first conduit has a generally uniform outer dimension in cross section along the length of the first conduit; and
the second conduit including a first end configured to couple with the first conduit and a second end joined to an external anchor, wherein the external anchor is configured to rest against an outer skin surface over a chest wall.

2. The airway bypass device of claim 1 wherein the outer dimension of the first conduit is smaller than a dimension in cross section of a passage extending through the second conduit, such that the second conduit slides into the first conduit.

3. The airway bypass device of claim 1 wherein the outer dimension of the second conduit is smaller than a dimension in cross section of a passage extending through the first conduit, such that the first conduit slides into the second conduit.

4. The airway bypass device of claim 1, further comprising an air passage extending through the first and second conduits, wherein the air passage includes an inlet opening open to the permeable structure and an outlet opening at the second end of a second of the second conduit.

5. The airway bypass device of claim 1, further comprising a lumen extending through the first and second conduits, wherein the lumen includes an inlet opening open to the permeable structure and an outlet opening at the second end of a second of the second conduit, and the lumen has a passage size sufficient to receive one or more of an endoscope, laparoscope, boroscope and bronchoscopes,

6. The airway bypass device of claim 1, wherein the first and second conduits are cylindrical structures and have common axes.

7. The airway bypass device of claim 1, wherein one of the first and second conduits has a textured outer surface configured to promote ingrown tissue growth.

8. The airway bypass device of claim 1, further comprising a cap releasably attached to a second end of the conduit.

9. The airway bypass device of claim 8 wherein the cap has a perimeter overlapping a perimeter of the external anchor.

10. The airway bypass device of claim 8, wherein the cap attaches to the second end of the conduit by at least one of a lock, groove engaging a detent, an O-ring seal, a magnetic coupling and a threaded male and female screw arrangement.

11. The airway bypass device of claim 8, wherein the cap is porous and forms a filter.

12. The airway bypass device of claim 1, wherein the anchor is an annular flange.

13. The airway bypass device of claim 1, wherein the anchor is an integral, single piece component with the second conduit.

14. The airway bypass device of claim 1, wherein the anchor includes apertures to receive sutures.

15. The airway bypass device of claim 1, wherein the first and second conduits telescope to elongate the airway bypass device.

16. The airway bypass device of claim 1, wherein the permeable structure comprises a scaffolding structure.

17. (canceled)

18. An implantable port configured to be implanted in a patient's chest wall to provide a passageway from lung parenchyma through the chest wall to atmosphere, the port comprising:
an permeable structure configured to be embedded in the lung parenchyma and defining within the structure an air capture chamber within the permeable structure, wherein the permeable structure includes at least one opening through which passes air from the lung tissue into the air capture chamber;
a first tube having outer dimension in cross section along the entire length of the first conduit which is smaller than an outer dimension of the permeable structure; and
a second conduit including a first end configured to couple with the first conduit and a second end joined to an external anchor, wherein the external anchor is configured to rest against an outer skin surface over a chest wall.

19. The implantable port of claim 18 wherein the outer dimension of the first conduit is substantially constant along a length of the conduit.

20. The implantable port of claim 18, further comprising an air passage extending through the first and second conduits, wherein the air passage includes an inlet opening open to the permeable structure and an outlet opening at the second end of a second of the second conduit.

21. The implantable port of claims of claim 18, further comprising a lumen extending through the first and second conduits, wherein the lumen includes an inlet opening open to the permeable structure and an outlet opening at the second end of a second of the second conduit, and the lumen has a passage size sufficient to receive one or more of an endoscope, laparoscope, boroscope and bronchoscopes,

22. The implantable port of claim 18, wherein the first and second conduits are cylindrical structures and have common axes.

23. The implantable port of claim 18, wherein one of the first and second conduits has a textured outer surface configured to promote ingrown tissue growth.

24. The implantable port of claim 18, further comprising a cap releasably attached to a second end of the conduit.

25. The implantable port of claim 24 wherein the cap has a perimeter overlapping a perimeter of the external anchor.

26. The implantable port of claim 24, wherein the cap attaches to the second end of the conduit by at least one of a lock, groove engaging a detent, an O-ring seal, a magnetic coupling and a threaded male and female screw arrangement.

27. The implantable port of claim 24, wherein the cap is porous and forms a filter.

28. The implantable port of claim 18, wherein the anchor is an annular flange.

29. The implantable port of claim 18, wherein the anchor is an integral, single piece component with the second conduit.

30. The implantable port of claim 18, wherein the anchor includes apertures to receive sutures.

31. The implantable port of claim 18, wherein the first and second conduits telescope to elongate the airway bypass device, and the elongation is selected to position the permeable structure in the lung tissue.

32. The implantable port of claim 18 wherein the permeable structure comprises a scaffolding structure, optionally a scaffolding of struts or fibers or wires.

33. (canceled)
